(19) United States
(12) Patent Application Publication
(10) Pub. No.: US 2023/0242637 A1
(43) Pub. Date: Aug. 3, 2023

(54) ANTI-SNAP 25 ANTIBODY FOR INHIBITING SNARE COMPLEX AND USE THEREOF

(71) Applicant: HAUUL BIO, Chuncheon-si, Gangwon-do (KR)

(72) Inventors: Hee-Jun NA, Chuncheon-si, Gangwon-do (KR); Yun-Suk LEE, Chuncheon-si, Gangwon-do (KR); Je-Ok YOO, Chuncheon-si, Gangwon-do (KR); Kwang-Soon LEE, Chuncheon-si, Gangwon-do (KR); Kangseung LEE, Chuncheon-si, Gangwon-do (KR); Seung Je MIN, Chuncheon-si, Gangwon-do (KR); Ji Won KANG, Gangneung-si, Gangwon-do (KR)

(73) Assignee: HAUUL BIO, Chuncheon-si, Gangwon-do (KR)

(21) Appl. No.: 17/768,874

(22) PCT Filed: Oct. 14, 2020
(86) PCT No.: PCT/KR2020/013967

§ 371 Date: Apr. 14, 2022

(30) Foreign Application Priority Data
Oct. 16, 2019 (KR) 10-2019-0128600

(51) Int. Cl. C07K 16/28; A61Q 19/00

(52) U.S. Cl. CPC C07K 16/28; A61Q 19/00; C07K 2317/565; C07K 2317/622

(57) ABSTRACT

The present disclosure relates to an anti-SNAP 25 antibody for inhibiting a SNARE complex, and a use thereof and, more specifically, to an anti-SNAP 25 antibody comprising heavy chain CDRs and light chain CDRs of specific sequences, or an antigen-binding fragment thereof. The anti-snap 25 antibody inhibits the formation of a SNARE complex, and thus is expected to be advantageously used for reducing or treating skin wrinkles.

### TECHNICAL FIELD

The present disclosure relates to an anti-SNAP 25 antibody for inhibiting a SNARE complex, and a use thereof.

### BACKGROUND ART

Membrane fusion taking place in cells is caused by proteins called soluble N-ethylmaleimide sensitive factor attachment protein receptor (SNARE). SNARE proteins refer to a group of specific proteins that are very well conserved across all species, and a SNARE complex refers to a complex of these proteins. SNARE protein is divided into target (t-) SNARE and vesicular (v-) SNARE, wherein t-SNARE refers to a SNARE protein present in the presynaptic membrane, and v-SNARE refers to a SNARE protein present in the synaptic vesicle. t-SNARE is composed of an integral membrane protein called Syntaxin 1A and a peripheral membrane protein called SNAP 25 (soluble NSF attachment protein of 25 kDa), and the functional unit is considered to be a complex thereof (t-SNARE complex). v-SNARE refers to a membrane protein called vesicle-associated membrane protein 2 (VAMP2 or synaptobrevin). These SNARE proteins have a region of about 60 to 70 aa called the ‘SNARE core’, and these regions come together to form a four-helical bundle called the SNARE complex.

Although studies on the SNARE complex are being actively conducted, studies on antibodies that inhibit the SNARE complex are still insufficient.

### DISCLOSURE

### Technical Problem

It is an object of the present disclosure to provide an anti-SNAP 25 antibody or antigen-binding fragment thereof for inhibiting a SNARE complex.

Another object of the present disclosure is to provide a fusion anti-SNAP 25 antibody or antigen-binding fragment thereof, in which a TAT peptide is additionally bound to the anti-SNAP 25 antibody or an antigen-binding fragment thereof.

Another object of the present disclosure is to provide a nucleic acid molecule encoding the antibody or an antigen-binding fragment thereof, a recombinant expression vector including the nucleic acid molecule, and cells transformed with the recombinant expression vector.

Another object of the present disclosure is to provide a composition for detecting SNAP 25 antigens, including the antibody or an antigen-binding fragment thereof as an active ingredient.

Another object of the present disclosure is to provide a cosmetic composition for preventing or ameliorating skin wrinkles, including the antibody or an antigen-binding fragment thereof as an active ingredient, a cosmetic composition for antioxidation, a health functional food composition for preventing or ameliorating skin wrinkles, and a pharmaceutical composition for preventing or treating skin wrinkles.

### Technical Solutions

To achieve the above objects, example embodiments of the present disclosure provide an anti-SNAP 25 antibody or antigen-binding fragment thereof, including a light chain variable region including a light chain CDR1 consisting of an amino acid sequence represented by SEQ ID NO: 1, a light chain CDR2 consisting of an amino acid sequence represented by SEQ ID NO: 2, and a light chain CDR3 consisting of an amino acid sequence represented by SEQ ID NO: 3, and a heavy chain variable region including a heavy chain CDR1 consisting of an amino acid sequence represented by SEQ ID NO: 4, a heavy chain CDR2 consisting of an amino acid sequence represented by SEQ ID NO: 5, and a heavy chain CDR3 consisting of an amino acid sequence represented by SEQ ID NO: 6.

In addition, example embodiments of the present disclosure provide a fusion anti-SNAP 25 antibody or antigen-binding fragment thereof in which a TAT peptide represented by in SEQ ID NO: 7 is additionally bound to the antibody or an antigen-binding fragment thereof.

Example embodiments of the present disclosure also provide a nucleic acid molecule encoding the antibody or an antigen-binding fragment thereof.

In addition, example embodiments of the present disclosure provide a recombinant expression vector including the nucleic acid molecule.

In addition, example embodiments of the present disclosure provide a cell transformed by the recombinant expression vector.

In addition, example embodiments of the present disclosure provide a composition for detecting SNAP 25 antigen, including the antibody or an antigen-binding fragment thereof as an active ingredient.

In addition, example embodiments of the present disclosure provide a cosmetic composition for preventing or ameliorating skin wrinkles, including the antibody or an antigen-binding fragment thereof as an active ingredient.

In addition, example embodiments of the present disclosure provide a cosmetic composition for antioxidation, including the antibody or an antigen-binding fragment thereof as an active ingredient.

In addition, example embodiments of the present disclosure provide a health functional food composition for preventing or ameliorating skin wrinkles, including the antibody or an antigen-binding fragment thereof as an active ingredient.

In addition, example embodiments of the present disclosure provide a pharmaceutical composition for preventing or treating skin wrinkles, including the antibody or an antigen-binding fragment thereof as an active ingredient.

### Advantageous Effects

Example embodiments of the present disclosure relate to an anti-SNAP 25 antibody for inhibiting a SNARE complex and a use thereof, and more particularly, to an anti-SNAP 25 antibody or antigen-binding fragment thereof including a heavy chain CDR and a light chain CDR of specific sequences. The anti-SNAP 25 antibody is expected to be usefully applied for ameliorating or treating skin wrinkles by inhibiting formation of a SNARE complex.

### BRIEF DESCRIPTIONS OF THE DRAWINGS

FIG. 1 shows a result of ELISA assay for screening anti-SNAP 25 scFv antibody.

FIG. 2 shows a result of the size and protein purity via SDS-PAGE after purification of a cell-permeable anti-SNAP 25 scFv protein.

FIG. 3 shows a result of native-PAGE analysis for SNARE complex formation inhibitory ability of a cell-permeable anti-SNAP 25 scFv protein.

FIG. 4 shows a result of inhibition of MMP-1 collagenase activity by a cell-permeable anti-SNAP 25 scFv protein.

FIG. 5 shows a result of cell permeability of a cell-permeable anti-SNAP 25 scFv protein via Western blot.

FIG. 6 shows results of pig skin permeability of a cell-permeable anti-SNAP 25 scFv protein via DAB staining.

### BEST MODE

An example embodiment of the present disclosure provides an anti-SNAP 25 antibody or antigen-binding fragment thereof, including a light chain variable region including a light chain CDR1 consisting of an amino acid sequence represented by SEQ ID NO: 1, a light chain CDR2 consisting of an amino acid sequence represented by SEQ ID NO: 2, and a light chain CDR3 consisting of an amino acid sequence represented by SEQ ID NO: 3; and a heavy chain variable region having a heavy chain CDR1 consisting of an amino acid sequence represented by SEQ ID NO: 4, a heavy chain CDR2 consisting of an amino acid sequence represented by SEQ ID NO: 5 and a heavy chain CDR3 consisting of an amino acid sequence represented by SEQ ID NO: 6.

In addition, an example embodiment of the present disclosure provides a fusion anti-SNAP 25 antibody or antigen-binding fragment thereof in which a TAT peptide represented by SEQ ID NO: 7 is additionally bound to the antibody or an antigen-binding fragment thereof.

Here, the CDRs consisting of amino acids represented by SEQ ID NO: 1 to SEQ ID NO: 6 are described in TABLE 1.

In addition, the amino acid sequence of the TAT peptide used in the present disclosure is “YGRKKRRQRRR” (SEQ ID NO: 7), and a base sequence of the TAT peptide is “TAT GGC CGC AAA AAA CGC CGC CAG CGC CGC CGC” (SEQ ID NO: 8).

In example embodiments of the present disclosure, the term “antibody” as used herein refers to a protein molecule which includes an immunoglobulin molecule having immunological reactivity with a specific antigen and serves as a receptor that specifically recognizes an antigen. For example, it may include all of a monoclonal antibody, a polyclonal antibody, a full-length antibody, and an antibody fragment. Also, the term “antibody” as used herein may include a bivalent or bispecific molecule (e.g., a bispecific antibody), a diabody, a triabody, or a tetrabody.

The term “monoclonal antibody” as used herein refers to an antibody molecule of a single molecular composition obtained from a group of substantially identical antibodies, and such monoclonal antibody exhibits single binding ability and affinity for a specific epitope unlike a polyclonal antibody capable of binding to multiple epitopes. The term “full-length antibody” as used herein has a structure composed of two full-length light chains and two full-length heavy chains, and each light chain is connected to the heavy chain by a disulfide bond. The heavy chain constant region has gamma (γ), mu (µ), alpha (α), delta (δ), and epsilon (ε) types as well as subclasses including gamma 1 (γ1), gamma 2 (γ2), gamma 3 (γ3), gamma 4 (γ4), alpha 1 (α1), and alpha 2 (α2). The constant region of the light chain has kappa (κ) and lambda (λ) types. IgG is a subtype including IgG1, IgG2, IgG3, and IgG4.

The term “heavy chain” as used herein may include both a full-length heavy chain and fragments thereof, wherein the full-length heavy chain is composed of a variable region VH having an amino acid sequence with a sequence of a variable region sufficient to give specificity to an antigen and three constant regions such as CH1, CH2 and CH3. In addition, the term “light chain” as used herein may include both a full-length light chain and fragments thereof, wherein the full-length light chain is composed of a variable region VL, which has an amino acid sequence with a sequence of a variable region sufficient to give specificity to an antigen, and a constant region CL.

The terms “fragment”, “antibody fragment” and “antigen-binding fragment” as used herein refer to any fragment of an antibody of an example embodiment of the present disclosure that retains the antigen-binding function of the antibody, and are used interchangeably. Exemplary antigen binding fragments include Fab, Fab′, F(ab′)2, and Fv, but are not limited thereto.

The antibody or antigen-binding fragment thereof in an example embodiment of the present disclosure may include not only the sequence of the antibody described herein, but also a biological equivalent thereof, within the range that the ability to specifically bind to SNAP 25 is derived. For example, additional modifications may be made to the amino acid sequence of the antibody to further improve binding affinity and/or other biological properties of the antibody. Such modifications include, for example, deletions, insertions, and/or substitutions of amino acid sequence residues of the antibody. Such variations in amino acid are made based on the relative similarity of amino acid side chain substituents, such as hydrophobicity, hydrophilicity, charge, and size. Through analysis on the size, shape, and type of amino acid side chain substituents, it was determined that arginine, lysine, and histidine are all positively charged residues; alanine, glycine, and serine have similar sizes; and phenylalanine, tryptophan and, tyrosine have similar shapes. Therefore, based on this, arginine, lysine, and histidine; alanine, glycine, and serine; and phenylalanine, tryptophan, and tyrosine may be considered to be biologically functional equivalents.

An example embodiment of the present disclosure also provides a nucleic acid molecule encoding the antibody or an antigen-binding fragment thereof.

The term “nucleic acid molecule” as used herein comprehensively includes DNA (gDNA and cDNA) and RNA molecules. In addition, nucleotides, which are basic structural units in nucleic acid molecules, include natural nucleotides as well as an analogue in which sugars or base sites are modified. The sequences of the nucleic acid molecules encoding the heavy and light chain variable regions of the present disclosure may be modified, and the modification includes additions, deletions, or non-conservative or conservative substitutions of nucleotides.

In addition, an example embodiment of the present disclosure also provides a recombinant expression vector including the nucleic acid molecule.

In an example embodiment of the present disclosure, the term “vector” as used herein refers to a self-replicating DNA molecule used to carry a clonal gene (or another piece of clonal DNA).

In an example embodiment of the present disclosure, the term “expression vector” as used herein refers to a recombinant DNA molecule including a desired coding sequence and an appropriate nucleic acid sequence essential for expressing a coding sequence operably linked in a specific host organism. The expression vector may preferably include one or more selective markers. The marker is a nucleic acid sequence that is selectable by a conventional chemical method and includes all genes capable of distinguishing a transformed cell from a non-transformed cell. Examples include genes resistant to antibiotics such as ampicillin, kanamycin, geneticin (G418), bleomycin, hygromycin, and chloramphenicol. However, the examples are not limited thereto and may be appropriately selected by those skilled in the art.

To express the DNA sequences of an example embodiment of the present disclosure, any of a wide variety of expression regulatory sequences may be used in the vector. Examples of useful expression regulatory sequences include, for example, early and late promoters of SV40 or adenovirus, promoters and enhancers of CMV, LTR of retrovirus, lac system, trp system, TAC or TRC system, T3 and T7 promoters, major operator and promoter regions of lambda phage, regulatory regions of fd-code proteins, promoters for 3-phosphoglycerate kinase or other glycolytic enzymes, promoters of the phosphatases such as Pho5, promoters of yeast alpha-breeding system, other different sequences of construction and induction known to regulate expression of genes in prokaryotic or eukaryotic cells or viruses thereof, and various combinations thereof.

As the vector expressing the antibody of an example embodiment of the present disclosure, a vector system in which the light chain and the heavy chain are co-expressed in a single vector or a system in which the light chain and the heavy chain are expressed in separate vectors respectively are all possible. In the latter case, both vectors are introduced into a host cell through co-transformation and targeted transformation. The co-transfomation is a method of selecting cells expressing both the light and heavy chains after simultaneously introducing each vector DNA encoding the light and heavy chains into a host cell. The targeted transformation is a method of selecting cells transformed with a vector including a light chain (or a heavy chain) and re-transforming the selected cells expressing the light chain with a vector including a heavy chain (or a light chain), and finally selecting cells expressing both the light chain and the heavy chain.

In addition, an example embodiment of the present disclosure provides a cell transformed with a recombinant expression vector.

Cells capable of stably, continuously cloning and expressing the vector of an example embodiment of the present disclosure may be any host cell known in the art, including, for example, Bacillus sp. strains such as Escherichia coli, Bacillus subtilis, and Bacillus thuringiensis or prokaryotic host cells such as Streptomyces, Pseudomonas (e.g., Pseudomonas putida), Proteus mirabilis, or Staphylococcus (e.g., Staphylococcus carnosus), but are not limited thereto.

In the method for producing the antibody or antigen-binding fragment thereof, culture of the transformed cells may be conducted in accordance with an appropriate medium and culture conditions known in the art. Such a culture process may be easily adjusted to be used by those skilled in the art depending on the selected strain. Cell culture is divided into suspension culture and adherent culture depending on the cell growth type, and also divided into batch, fed-batch, and continuous culture methods depending on the culture method. The medium used for culture should suitably satisfy the requirements of a particular strain.

In addition, an example embodiment of the present disclosure provides a composition for detecting SNAP 25 antigen, including the antibody or antigen-binding fragment thereof as an active ingredient.

In addition, an example embodiment of the present disclosure provides a cosmetic composition for preventing or ameliorating skin wrinkles, including the antibody or antigen-binding fragment thereof as an active ingredient. Specifically, the composition may inhibit formation of a SNARE complex.

In addition, an example embodiment of the present disclosure provides a cosmetic composition for antioxidation including the antibody or antigen-binding fragment thereof as an active ingredient. Specifically, the composition may inhibit the production of reactive oxygen.

The cosmetic composition may include, in addition to the active ingredient, conventional adjuvants, such as stabilizers, solubilizers, vitamins, pigments, and fragrances, and carriers.

The formulation of the cosmetic composition may be prepared in any formulation conventionally prepared in the art, and may have a formulation selected from the group consisting of external skin ointment, cream, softening toner, nourishing toner, pack, essence, hair tonic, shampoo, conditioner, hair conditioner, hair treatment, gel, skin lotion, skin softener, skin toner, astringent, lotion, milk lotion, moisture lotion, nourishing lotion, massage cream, nourishing cream, eye cream, moisture cream, hand cream, foundation, nourishing essence, sunscreen, soap, cleansing foam, cleansing lotion, cleansing cream, body lotion, and body cleanser, but is not limited thereto. The composition of each of these formulations may contain various bases and additives necessary and appropriate to formulate the formulation, and the types and amounts of these components may be easily selected by those skilled in the art.

When the formulation is a paste, cream or gel, animal oil, vegetable oil, wax, paraffin, starch, tragacanth, cellulose derivatives, polyethylene glycol, silicone, bentonite, silica, talc, or zinc oxide may be used as a carrier component.

When the formulation is powder or a spray, lactose, talc, silica, aluminum hydroxide, calcium silicate, or polyamide powder may be used as the carrier component, and in particular, in the case of the spray, a booster such as chlorofluorohydrocarbon, propane/butane, or dimethyl ether may additionally be included.

When the formulation is a solution or an emulsion, solvents, solubilizers, or emulsifiers are used as the carrier component, including, for example, water, ethanol, isopropanol, ethyl carbonate, ethyl acetate, benzyl alcohol, benzyl benzoate, propylene glycol, 1,3-butylglycol oil, glycerol fatty ester, polyethylene glycol, or fatty acid ester of sorbitan.

When the formulation is a suspension, a liquid diluent such as water and ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, or tragacanth may be used as the carrier component.

In addition, an example embodiment of the present disclosure provides a health functional food composition for preventing or ameliorating skin wrinkles, including the antibody or antigen-binding fragment thereof as an active ingredient. Specifically, the composition may inhibit formation of the SNARE complex.

The health functional food composition may be provided in the form of powder, granules, tablets, capsules, syrups, beverages, or pills. In addition, the health food composition is used together with other food or food additives in addition to the composition according to an example embodiment of the present disclosure as an active ingredient and may be appropriately used according to a conventional method. The mixed amount of the active ingredient may be suitably determined depending on the purpose of use, for example, prevention, health control, or therapeutic treatment.

The effective dose of the antibody or antigen-binding fragment thereof contained in the health functional food composition may be allowed according to the effective dose of the pharmaceutical composition, but in the case of long-term intake for health and hygiene or for health control may be less than the above range. However, it is certain that the active ingredient may be used in an amount exceeding the range since there is no problem in terms of safety.

The type of health food is not particularly limited, and examples include meat, sausage, bread, chocolate, candy, snacks, confectionery, pizza, ramen, other noodles, gum, dairy products including ice cream, various soups, beverages, tea, drinking agents, alcoholic beverages, and vitamin complexes.

In addition, an example embodiment of the present disclosure provides a pharmaceutical composition for preventing or treating skin wrinkles including the antibody or antigen-binding fragment thereof as an active ingredient. Specifically, the composition may inhibit formation of a SNARE complex.

The pharmaceutical composition of an example embodiment of the present disclosure may further include a pharmaceutically acceptable carrier, and the pharmaceutically acceptable carrier is commonly used in the formulation, including lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methylcellulose, methylhydroxybenzoate, propylhydroxybenzoate, talc, magnesium stearate, and mineral oil, but is not limited thereto. A composition of an example embodiment of the present disclosure may further include lubricants, wetting agents, sweeteners, flavoring agents, emulsifiers, suspending agents, and preservatives, in addition to the above components.

The pharmaceutical composition of an example embodiment of the present disclosure may be administered orally or parenterally, and in the case of parenteral administration, administration may be conducted via intravenous injection, subcutaneous injection, intramuscular injection, intraperitoneal injection, endothelial administration, topical administration, intranasal administration, intrapulmonary administration, and rectal administration. In the case of oral administration, since protein or peptide is digested, oral compositions may be prepared by coating active agents or formulated to secure protection from degradation in the stomach, and the composition of an example embodiment of the present disclosure may be administered by any device capable of transporting an active ingredient to a target cell.

A suitable dosage of the pharmaceutical composition of an example embodiment of the present disclosure varies depending on elements such as formulation methods, administration methods, age, weight, sex, pathological condition, food, administration time, administration route, excretion rate, and response sensitivity of a patient. Thus, a physician with ordinary skill may determine and prescribe an effective dosage with ease for the desired treatment or prevention.

The pharmaceutical composition of an example embodiment of the present disclosure is formulated in a unit dosage form by using a pharmaceutically acceptable carrier and/or excipient according to a method that can be easily carried out by a person of ordinary skill in the art to which the present disclosure pertains, or it may be prepared by being introduced into a multi-dose container. In this case, the formulation may be in the form of a solution, a suspension, or an emulsion in oil or an aqueous medium, or may be in the form of an extract, powder, a discutient, a powder agent, a granule, a tablet, or a capsule agent. A dispersant or a stabilizer may additionally be included as well.

The composition of an example embodiment of the present disclosure may be administered as an individual therapeutic agent or in combination with other therapeutic agents and may be administered sequentially or simultaneously with conventional therapeutic agents.

### MODES FOR CARRYING OUT INVENTION

Hereinafter, the present disclosure will be described in more detail through examples. The examples are merely for illustrating the present disclosure in more detail, and it will be apparent to those of ordinary skill in the art that the scope of the present disclosure is not limited by the examples according to the gist of the present disclosure.

### <Example 1> Screening of Anti-SNAP 25 scFv Antibody

### 1. Implementation of Bio-Panning

Bio-panning was performed using an OPAL library with a diversity of 7.6 × 10⁹. 4 µg of SNAP 25 antigen was immobilized on an epoxy magnetic bead and reacted with input phage. The output titer was measured by elution of the phage reacted with the antigen. Information on the bio-panning was obtained by measuring the input and output titers every number of times, and status whether the operation is normally performed was checked. The amount of input phage used therefor was less than or equal to 4 × 10¹² cfu/mL (4 × 10¹² cfu/mL ≥) for each operation. The progress of the bio-panning was determined by output (1^{st} - 5 × 10⁶ cfu/mL, 2^{nd} - 1.5 × 10⁸ cfu/mL, and 3^{rd} - 1.7 × 10⁷ cfu/mL).

### 2. ELISA Assay

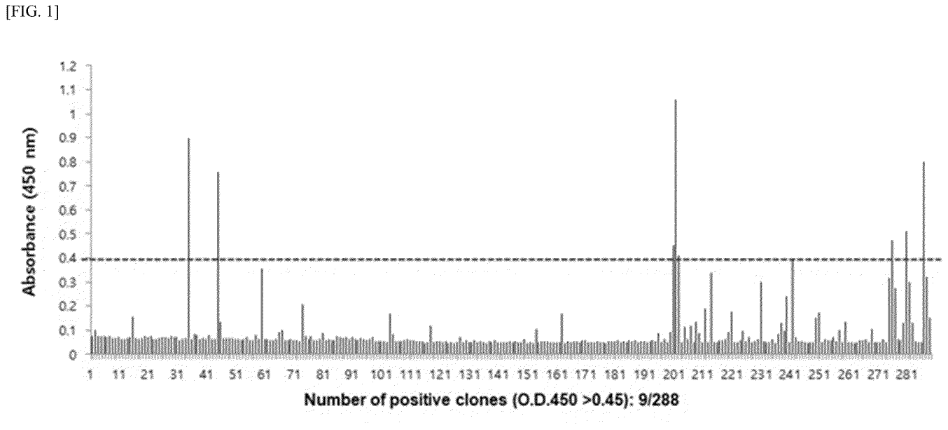

An ELISA assay was conducted for screening highly sensitive, highly specific antibodies. E. coli was infected with the obtained phages and spread on an LB plate to which antibiotics were added. After culturing in an incubator at 30° C. for 16 hours, the produced colonies were randomly collected. After each colony was cultured in the LB medium, IPTG was treated to induce expression of scFv, and E. coli was lysed to obtain a soluble fraction, followed by ELISA. First, 1 µg/mL of SNAP 25 recombinant protein was immobilized in a 96-well ELISA plate, and blocking was performed with PBS containing 1% BSA. After 1 hour, the cell lysate obtained above was treated and reacted at 4° C. for 16 hours. After washing the plate three times with PBS containing 0.1% Tween20, HRP-conjugated anti-HA antibody was diluted in a blocking solution at a ratio of 1:1000, followed by a reaction at room temperature for 1 hour. After washing the plate five times with PBS containing 0.1% Tween20, the color was developed with a TMB substrate to measure scFv antibody bound to the antigen via an ELISA reader. FIG. 1 shows a result of ELISA performed using SNAP 25. 288 antibodies were analyzed, and 9 types were screened, wherein OD 0.4 was arbitrarily set as a positive guideline and OD 0.1 as a negative guideline (FIG. 1).

### <Example 2> Sequencing of Anti-SNAP 25 scFv Antibody

Individual clones were screened through sequencing on positive clones of the 9 types of anti-SNAP 25 screened through the ELISA assay. The sequencing was required since there was a possibility that overlapping clones exist among the positive clones that were previously screened. As a result of sequencing, in the case of anti-SNAP 25, it was determined that 6 out of 9 positive clones were independent clones.

### <Example 3> Preparation of Cell-Permeable Anti-SNAP 25 scFv

### 1. Preparation of a TAT-Anti-SNAP 25 scFv Construct

A PCR product of one type of previously screened anti-SNAP 25 scFv (TABLE 1) was prepared using a primer, wherein a restriction enzyme site and TAT were inserted into the PCR product. 30 µL of each PCR product was treated with 4 µL of buffer 3.1, 1 µL of Sal I, 1 µL of Xho I, and 4 µL of distilled water, and a reaction was carried out for one hour at 37° C. After that, DNA was isolated to secure an insert to be inserted into the vector. pET28a (+) vector was treated for transformation in DH5α competent cells which were then spread on a kanamycin-added LB plate and incubated at 37° C. for 16 hours. The next day, colonies were collected and inoculated in the kanamycin-added LB media, followed by culture at 37° C. for 16 hours. The next day, after isolation of the vector using a mini-prep kit, 30 µL of the vector was treated with 4 µL of buffer 3.1, 1 µL of Sal I, 1 µL of Xho I, 1 µL of CIP, and 3 µL of distilled water and reacted at 37° C. for 1 hour, followed by purification. The concentrations of the purified vector and insert were measured with nanodrops, and ligation was performed at room temperature for 16 hours using T4 ligase for each ratio of the vector and the insert. After termination of ligation, transformation was performed in the DH5α competent cells which was then spread on the kanamycin-added LB plate and cultured at 37° C. for 16 hours. The next day, colonies were collected and inoculated in the kanamycin-added LB media, followed by culture at 37° C. for 16 hours. The next day, to check the insertion of the insert, plasmids were isolated, restriction was performed with Sal I and Xho I, and a band was observed by electrophoresis on 1% agarose gel.

TABLE 1

| Target | Clone | Light chain CDR sequence | | | Heavy chain CDR sequence | | |
|---|---|---|---|---|---|---|---|
| | | CDR1 | CDR2 | CDR3 | CDR1 | CDR2 | CDR3 |
| SNAP 25 | 5 | SVSSSNIGSNSVS | SNTH | GTWDYSLSA | NY AMS | AISSGGGNI | KNRIFDY |

### 2. Purification of TAT-Anti-SNAP 25 scFv Antibody

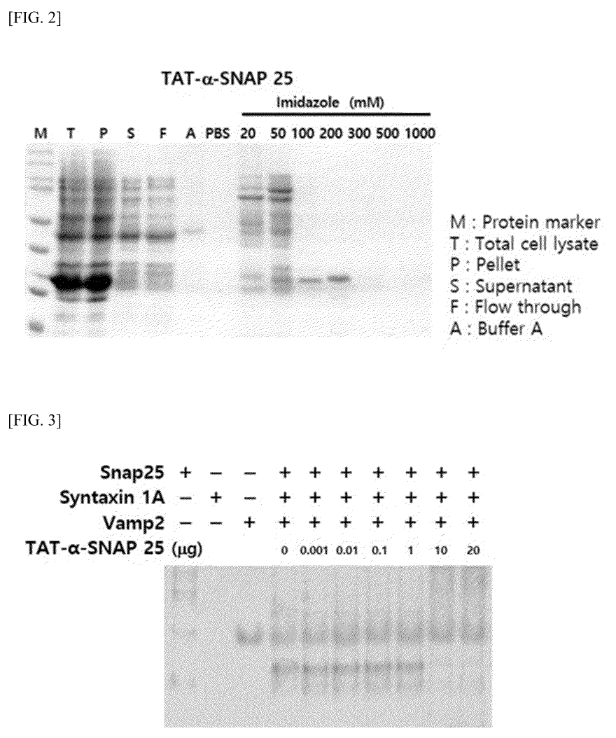

The cloned TAT-anti-SNAP 25 scFv antibody clone was treated for transformation in a BL21(DE3) E. coli host. Transformants were cultured in the kanamycin-containing LB media until OD₆₀₀ value became 0.6, and expression was induced by treating 0.5 mM of IPTG at 16° C. Cells obtained by centrifugation of the culture medium were suspended in a lysis buffer (50 mM of Tris-HCl, pH 7.5, 150 mM of NaCl), disrupted using an ultrasonicator, and then centrifuged to obtain a supernatant. The TAT-anti-SNAP 25 scFv protein was purified using a resin having affinity for Ni-NTA, and the size of the protein was determined to be about 30 kDa through SDS-PAGE analysis (FIG. 2).

### <Example 4> Cytotoxicity Test for Cell-Permeable Anti-SNAP 25 scFv

To determine the cytotoxicity against TAT-anti-SNAP 25 scFv antibody, mammalian neuronal PC12 cells were cultured in an incubator in the presence of CO₂ at 37° C. After the culture, the TAT-anti-SNAP 25 scFv antibody was treated by concentration and then further cultured under the same culture conditions. After the culture by treating 3-(4,5-dimethylthiazol-2-yl)-2,5-diphenyltetrazolium bromide (MTT) solution, the culture medium was removed, and dimethyl sulfoxide (DMSO) was added and mixed thoroughly. The absorbance was measured at 570 nm with ELISA Reader system. The values were shown in TABLE 2. As shown in TABLE 2, when TAT-anti-SNAP 25 scFv was treated at concentrations of 0.01, 0.1, 1, 10, 50, and 100 ppm, changes in cell shape and cell viability of mammalian neuronal cells were barely observed up to concentration of 100 ppm. Therefore, it was found that TAT-anti-SNAP 25 scFv is a substance with no cytotoxicity up to concentration of 100 ppm.

TABLE 2

Example 4
TAT-anti-SNAP 25

| Concentration (ppm) | Average | Error |
|---|---|---|
| 0 | 100.0 | 2.4 |
| 0.01 | 104.7 | 1.3 |
| 0.1 | 96.1 | 4.1 |
| 1 | 116.1 | 6.9 |
| 10 | 140.9 | 7.9 |
| 50 | 137.1 | 2.7 |
| 100 | 136.4 | 8.7 |

### <Example 5> Experiment for Ability to Inhibit Formation of a SNARE Complex by Cell-Permeable Anti-SNAP 25 scFv

To determine whether the TAT-anti-SNAP 25 scFv antibody inhibits formation of a SNARE complex, native-PAGE analysis was performed. When SNAP25, syntaxin 1A, and VAMP2 protein (LSbio) which are SNARE proteins were mixed at a concentration of 1:1:1 (1 µg), respectively, determined was whether the complex formed was inhibited by the addition of the TAT-anti-SNAP 25 scFv antibody. 1 µg of each protein was added, and each TAT-anti-SNAP 25 scFv antibody was treated by each concentration (0.001, 0.01, 0.1, 1, 10, 20 µg), thoroughly mixed, and then reacted at 4° C. for 1 hour. At the termination of the reaction, the reaction was stopped by adding a sample buffer, and then the formation of the SNARE complex was checked on 10% native-PAGE (FIG. 3). As a result, it was found that TAT-anti-SNAP 25 scFv effectively inhibited formation of the SNARE complex.

### <Example 6> Evaluation of Inhibition of MMP-1 Expression by Cell-Permeable Anti-SNAP 25 scFv

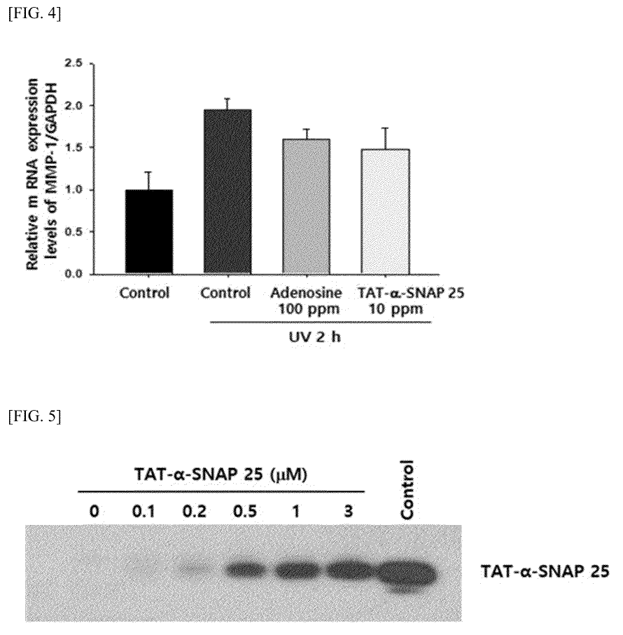

To determine the effect of TAT-anti-SNAP 25 scFv on collagen production, the effect of inhibiting MMP-1 activity by TAT-anti-SNAP 25 scFv was tested using Real Time PCR method. Mammalian neuronal PC12 cells were cultured in an incubator in the presence of CO₂ at 37° C. After the culture, UVA was irradiated using a UV irradiator system. Thereafter, 10 ppm of TAT-anti-SNAP 25 scFv antibody and 100 ppm of adenosine as a positive control were treated and then further cultured under the same culture conditions. After the culture, the cells were collected with 300 µL of TRIzol, transferred to a 1.5 mL tube, added and mixed with 50 µL of chloroform, and left at room temperature for 5 minutes. Then, centrifugation was performed at 15,000 rpm and 4° C. for 15 minutes. The supernatant was transferred to a new tube, mixed with the same amount of 2-propanol, left at room temperature for 5 minutes, and then centrifuged at 12,000 rpm and 4° C. for 20 minutes. After centrifugation, 2-propanol was discarded, and 300 µL of 75% ethanol was added, followed by centrifugation at 10,000 rpm and 4° C. for 10 minutes. 75% ethanol was discarded, and RNA pellet was dried at room temperature to remove the remaining ethanol. 30 µL of DEPC-treated purified water was added to the pellet to dissolve the same, and quantification was conducted at 260 nm. RT-PCR was performed using 1 µg of total RNA and TOPscript RT dry mix. MMP-1 and GAPDH used in Real Time PCR were synthesized in Macrogen to be used, and base sequences were shown in TABLE 3 below. Real Time PCR was performed using TOPreal™ Qpcr 2X PreMIX. As shown in FIG. 4, TAT-anti-SNAP 25 scFv inhibited MMP-1 collagenase, which is responsible for increased wrinkle formation by UV light. When treated with 10 ppm of TAT-anti-SNAP 25 scFv, an effect similar to a case that 100 ppm of a control was treated was derived.

TABLE 3

| | | |
|---|---|---|
| Primer | Sequence | |
| MMP-1 | Forward | 5′-ACGCAGATTTAGCCTCCGAA-3′ |
| | Reverse | 5′-TGACTTGGTAATGGGTTGCC-3′ |
| GAPDH | Forward | 5′-GACATGCCGCCTGGAGAAAC-3′ |
| | Reverse | 5′-AGCCCAGGATGCCCTTTAGT-3′ |

### <Example 7> Analysis of Cell and Skin Permeation of Cell-Permeable Anti-SNAP 25 scFv

### 1. Analysis of Cell Permeation of TAT-Anti-SNAP 25 scFv

To determine cell permeation of TAT-anti-SNAP 25 scFv, PC12 cells were cultured in a 6-well plate, replaced with 1.5 mL of fresh FBS-free culture solution, and treated with various concentrations of TAT-anti-SNAP 25 scFv. 1 hour later after the treatment, the cells were sufficiently washed with PBS and then collected to observe the degree of intracellular permeation of TAT-anti-SNAP 25 scFv by Western blot analysis. In order to determine whether the intracellularly permeated TAT-anti-SNAP 25 scFv was observed at a correct site (size), purely purified TAT-anti-SNAP 25 scFv (control) was also electrophoresed. As shown in FIG. 5, it was found that intracellular permeation of TAT-anti-SNAP 25 scFv took place effectively in a concentration-dependent manner.

### 2. Analysis of Skin Permeation of TAT-Anti-SNAP 25 scFv

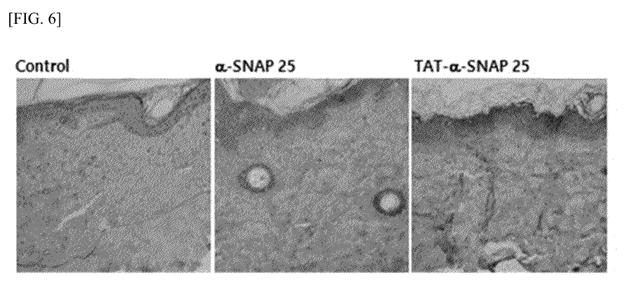

To determine skin permeation of TAT-anti-SNAP 25 scFv, tissue staining was performed using pig skin. Pig skin was treated with 10 µg of TAT-anti-SNAP 25 scFv and cultured in an incubator in the presence of CO₂ at 37° C. for 16 hours. After fixing pig skin with 4% formaldehyde, a paraffin block was prepared, and the tissue was excised by 5 µm using a microtome. Thereafter, the tissue was mounted on a slide, the paraffin was removed, and the tissue pieces were blocked for 30 minutes after hydration. Anti-His HRP antibody was reacted with the blocked tissue at room temperature for 1 hour. After washing with PBST, a reaction was performed in a DAB-chromogen solution for 5 minutes, followed by observation with an optical microscope after staining by H&E staining technique. FIG. 6 is results of comparing skin permeability of anti-SNAP 25 scFv and TAT-anti-SNAP 25 scFv. Whereas the anti-SNAP 25 scFv was not delivered into the pig skin tissues at all, it was found that an efficient skin delivery ability was secured in the case of TAT-anti-SNAP 25 scFv.

### <Example 8> Experiment for Reactive Oxygen Inhibitory Effect of Cell-Permeable Anti-SNAP 25 scFv

To determine intracellular reactive oxygen species (ROS) inhibitory effect of TAT-anti-SNAP 25 scFv antibody, mammalian neuronal PC12 cells were treated with TAT-anti-SNAP 25 scFv antibody, and then the content of intracellular reactive oxygen species was measured by fluorescence staining. PC12 cells inoculated in 96-well plates were cultured in an incubator in the presence of CO₂ at 37° C. and then treated with TAT-anti-SNAP 25 scFv antibody (10, 20, 50, 100 ppm). After the culture for 2 hours, 0.2 mM of hydrogen peroxide was added to each well, followed by additional culture under the same culture conditions. 20 µM of carboxy-H₂DCF-DA was added and reacted at 37° C. for 30 minutes. After sufficiently washing with PBS, 100 µL of PBS was re-added, and fluorescence was measured at 485/525 nm using ELISA Reader system. The values were shown in TABLE 4. As shown in TABLE 4, it was found that TAT-anti-SNAP 25 scFv effectively inhibited ROS generation.

TABLE 4

| Sample | Concentration (ppm) | ROS (fold of control) | | | |
|---|---|---|---|---|---|
| | | non treat control | | 0.2 mM H₂O₂ | |
| | | Average | Error | Average | Error |
| TAT-anti-SNAP 25 scFv | 0 | 1.000 | 0.136 | 1.485 | 0.097 |
| | 10 | 0.800 | 0.140 | 1.079 | 0.101 |
| | 20 | 0.857 | 0.071 | 1.069 | 0.185 |
| | 50 | 0.723 | 0.084 | 0.937 | 0.095 |
| | 100 | 0.649 | 0.039 | 0.861 | 0.059 |

As the specific parts of the present disclosure have been described in detail above, for those of ordinary skill in the art, these specific techniques are only preferred embodiments, and it is clear that the scope of the present disclosure is not limited thereto. Accordingly, the substantial scope of the present disclosure will be defined by the appended claims and equivalents thereof.

1. An anti-SNAP 25 antibody or antigen-binding fragment thereof, comprising a light chain variable region comprising a light chain CDR1 consisting of an amino acid sequence represented by SEQ ID NO: 1, a light chain CDR2 consisting of an amino acid sequence represented by SEQ ID NO: 2, and a light chain CDR3 consisting of an amino acid sequence represented by SEQ ID NO: 3; and a heavy chain variable region comprising a heavy chain CDR1 consisting of an amino acid sequence represented by SEQ ID NO: 4, a heavy chain CDR2 consisting of an amino acid sequence represented by SEQ ID NO: 5, and a heavy chain CDR3 consisting of an amino acid sequence represented by SEQ ID NO: 6.

2. A fusion anti-SNAP 25 antibody or antigen-binding fragment thereof, in which a TAT peptide represented by in SEQ ID NO: 7 is additionally bound to the antibody of claim 1 or an antigen-binding fragment thereof.

3-6. (canceled)

7. A method of preventing or ameliorating skin wrinkles, comprising;
providing a cosmetic composition comprising the antibody or an antigen-binding fragment thereof of claim 1 as an active ingredient; and
administering the cosmetic composition to a subject.

8-12. (canceled)

13. A method of preventing or ameliorating skin wrinkles, comprising:
providing a cosmetic composition comprising the antibody or antigen-binding fragment thereof of claim 2 as an active ingredient; and
administering the cosmetic composition to a subject.


SEQUENCE LISTING (12 sequences)

SEQ ID NO: 1 - protein, 13 aa, Artificial Sequence
  light chain CDR1
SVSSSNIGSNSVS

SEQ ID NO: 2 - protein, 4 aa, Artificial Sequence
  light chain CDR2
SNTH

SEQ ID NO: 3 - protein, 9 aa, Artificial Sequence
  light chain CDR3
GTWDYSLSA

SEQ ID NO: 4 - protein, 5 aa, Artificial Sequence
  heavy chain CDR1
NYAMS

SEQ ID NO: 5 - protein, 9 aa, Artificial Sequence
  heavy chain CDR2
AISSGGGNI

SEQ ID NO: 6 - protein, 8 aa, Artificial Sequence
  heavy chain CDR3
KNRINFDY

SEQ ID NO: 7 - protein, 11 aa, Artificial Sequence
  TAT peptide
YGRKKRRQRRR

SEQ ID NO: 8 - DNA, 33 bp, Artificial Sequence
  TAT peptide
tatggccgcaaaaaacgccgccagcgccgccgc

SEQ ID NO: 9 - DNA, 20 bp, Artificial Sequence
  Primer (MMP-1 Forward)
acgcagatttagcctccgaa

SEQ ID NO: 10 - DNA, 20 bp, Artificial Sequence
  Primer (MMP-1 Reverse)
tgacttggtaatgggttgcc

SEQ ID NO: 11 - DNA, 20 bp, Artificial Sequence
  Primer (GAPDH Forward)
gacatgccgcctggagaaac

SEQ ID NO: 12 - DNA, 20 bp, Artificial Sequence
  Primer (GAPDH Reverse)
agcccaggatgccctttagt